(19) United States
(12) Patent Application Publication
(10) Pub. No.: US 2015/0010438 A1
(43) Pub. Date: Jan. 8, 2015

(54) MOBILE CUSTOM-MADE HAND-HELD CHEMICAL DETECTION DEVICE FOR INTERFACING WITH A SMART DEVICE

(71) Applicants: Iem Heng, Floral Park, NY (US); Shaojin Zhang, Brooklyn, NY (US)

(72) Inventors: Iem Heng, Floral Park, NY (US); Shaojin Zhang, Brooklyn, NY (US)

(21) Appl. No.: 13/936,314

(22) Filed: Jul. 8, 2013

(51) Int. Cl. G01N 33/00

(52) U.S. Cl. CPC G01N 33/004; G01N 33/0073; G01N 33/0062
USPC 422/98

(57) ABSTRACT

A mobile custom-made hand-held chemical detection device interfacing with a smart device. The device includes at least one sensor, a microcontroller, and a Bluetooth module. The at least one sensor detects an associated chemical and generates information in response thereto so as to form chemical detection information. The microcomputer is operatively connected to the at least one sensor and processes the chemical detection information therefrom so as to form processed chemical detection information. The Bluetooth module is operatively connected to the microcontroller and the smart device, and communicates the processed chemical detection information from the microcontroller to the smart device for interpretation.

### 1. CROSS REFERENCE TO RELATED APPLICATIONS

The instant non-provisional patent application claims priority from provisional patent application No. 61/690,845, filed on Jul. 6, 2012, for a HANDHELD MONITOR FOR REMOTE CHEMICALS, and incorporated herein in its entirety by reference thereto.

### 2. BACKGROUND OF THE INVENTION

A. Field of the Invention

The embodiments of the present invention relate to a chemical detection device, and more particularly, the embodiments of the present invention relate to a mobile custom-made hand-held chemical detection device for interfacing with a smart device.

B. Description of the Prior Art

Over the years chemical gases in the U.S. and around the world have caused and taken many innocent lives, which could have been prevented. Of primary concern are the human health effects of chemical gas including premature mortality and chronic illnesses, such as bronchitis and asthma. Despite the tremendous economic costs and pervasive negative health impacts of bad chemical gas, chemical gas often goes unnoticed because it is largely invisible. Much of what happens in our immediate environment passes without being noticed by the public despite the fact that there are recording and crowdsourcing devices installed in some neighborhoods, which monitor air quality.

A mobile custom-made hand-held chemical detection (“CHCD”) device of the embodiments of the present invention captures a spectrum of lost reality and returns it to the users in real time as the events unfold. By making these specific environmental events available to participants in real time and location, the mobile CHCD device of the embodiments of the present invention supplements the qualitative information reported by government agencies, with quantitative information obtained from hand-held sensing devices that observe and record aspects of the environments that are either impossible to perceive directly, e.g., pollutant gas concentrations, or difficult to quantify and communicate in a consistent manner.

The mobile CHCD device of the embodiments of the present invention allows individuals to broadcast what is happening with their environment, crowdsource their own information with that from other participants, and identify patterns and commonalities. Thus, the mobile CHCD device of the embodiments of the present invention makes the detection of chemical gas possible by concerned citizens, thereby empowering communities to advocate for healthy environments.

Numerous innovations for wireless devices for detecting, and/or identifying, and/or monitoring, and/or warning, and/or notifying remote environmental conditions have been provided in the prior art, which will be described below in chronological order to show advancement in the art, and which are incorporated herein in their entireties by reference thereto. Even though these innovations may be suitable for the specific individual purposes to which they address, nevertheless, they differ from the embodiments of the present invention.

(1) U.S. Pat. No. 6,023,223 to Baxter, Jr.

U.S. Pat. No. 6,023,223—issued to Baxter, Jr. on Feb. 8, 2000 in U.S. class 340 and subclass 531—teaches an early warning detection and notification network for monitoring environmental conditions. The network includes a plurality of remotely located environmental sensors having a communications uplink to one or more earth orbiting satellites or other wireless transmission apparatus, a downlink interface to a database server having one or more data tables holding environmental data, and a communications interface between the database server and the Internet. The sensors periodically upload environmental condition data to the satellite. The satellite downloads the condition data to the database server. The communications interface provides access to the condition data through the Internet. End-users access the system through the Internet and retrieve real-time data on various environmental conditions based on a database query. The query results are groupable by the geographic region, the type of environmental data, or a combination of both. End-users may also employ preset trigger levels for certain environmental conditions. When the trigger levels are exceeded, the end-user is notified by email, pager, automated voice response, or the like.

(2) U.S. Pat. No. 6,356,625 B1 to Castellani et al.

U.S. Pat. No. 6,356,625 B1—issued to Castellani et al. on Mar. 12, 2002 in U.S. class 379 and subclass 32.01—teaches an environment monitoring telephone network system including a plurality of environment parameters detecting and transmitting units that transmit data toward a remote acquisition exchange. The units are provided with analog input sensors, at least a telephone network private line in order to transmit detected data to the remote acquisition exchange where a Data Logger is located and to supply remotely the detecting and transmitting unit, and a plurality of receiving units, each interconnected with a specific detecting and transmitting unit. The receiving units are located at the remote acquisition exchange and are linked with the input channels of a Data Logger that in turn is linked, by a modem to a storing and processing center through a switched telephone network.

(3) United States Patent Application Publication Number US 2004/0111232 A1 to Butler et al.

United States Patent Application Publication Number US 2004/0111232 A1—published to Butler et al. on Jun. 10, 2004 in U.S. class 702 and subclass 130—teaches a method of generating a temperature-compensated absorbance spectrum. The method includes the steps of providing a sample spectrum and an estimated temperature of a backdrop object from a set of known temperature spectra related to a known background temperature, selecting at least two known temperature spectra representing a background temperature above and below the estimated temperature, comparing the sample spectrum to the known temperature spectra in order to determine a sample background spectrum, and calculating an absorbance spectrum from the sample spectrum and the background spectrum.

(4) U.S. Pat. No. 6,946,671 B2 to Smith et al.

U.S. Pat. No. 6,946,671 B2—issued to Smith et al. on Sep. 20, 2005 in U.S. class 250 and subclass 559.4—teaches a system and method for identifying, reporting, and evaluating a presence of a solid, liquid, gas, or other substance of interest, particularly, a dangerous, hazardous, or otherwise threatening chemical, biological, or radioactive substance. The system includes one or more substantially automated and location self-aware remote sensing units, a control unit, and one or more data processing and storage servers. Data is collected by the remote sensing units and transmitted to the control unit. The control unit generates, and uploads a report incorporating the data, to the servers and thereafter the report is available for review by a hierarchy of responsive and evaluative authorities via a wide area network. The evaluative authorities include a group of relevant experts who may be widely, or even globally, distributed.

(5) United States Patent Application Publication Number US 2008/0287144 A1 to Sabata et al.

United States Patent Application Publication Number US 2008/0287144 A1 published to Sabata et al. on Nov. 20, 2008 in U.S. class 455 and subclass 456.6 teaches a system and method that use mobile sensor nodes for monitoring of mobile assets. One or more mobile sensor nodes have mobility that is unpredictable to a wireless network. The mobile sensor nodes collect data with the goal of monitoring the environment, including weather, pollution, biological and chemical agents for security application, and traffic application. The mobile sensors form local groups opportunistically to coordinate measurements and other actions. The collected sensor data is stored locally with global positioning system (GPS) data and time of data collected. The information is transmittable to a remote computer opportunistically using a low cost method, such as WiFi or a cell phone network. The information is aggregated to provide environmental maps, traffic maps, and maps to first responders in the event of biological, nuclear, or chemical calamity.

(6) U.S. Pat. No. 8,041,834 B2 to Ferri et al.

U.S. Pat. No. 8,041,834 B2—issued to Ferri et al. on Oct. 18, 2011 in U.S. class 709 and subclass 238—teaches a system and method for implementing a wireless sensor network. The system includes a plurality of motes. Each mote has a sensor and a wireless communication system for communicating with neighboring motes, a distributed routing table distributed amongst each of the plurality of motes, and an update system for periodically updating the distributed routing table.

(7) U.S. Pat. No. 8,150,465 B2 to Zhang et al.

U.S. Pat. No. 8,150,465 B2—issued to Zhang et al. on Apr. 3, 2012 in U.S. class 455 and subclass 557—teaches sensors mounted on vehicles, e.g., buses, taxis, police cars, and public personnel, e.g., policemen, are used to monitor various conditions and situations, such as air quality, potential biological and chemical attacks, and road and traffic conditions. A method for estimating the number of mobile sensors required to cover a region of interest also is taught. A relatively small number of mobile sensors may be sufficient to cover a large area at a lower cost and less complexity than a fixed sensor network.

(8) United States Patent Application Publication Number US 2012/0102165 A1 to Gruen et al.

United States Patent Application Publication Number US 2012/0102165 A1 published to Gruen et al. on Apr. 26, 2012 in U.S. class 709 and subclass 222—teaches a method, system, and computer program product for deploying a location-based application providing crowdsourced structured points of input for data entry. In an embodiment, a method for deploying a location-based application providing crowdsourced structured points of input for data entry includes the selection of a location-based application component, such as a map, for inclusion in a deployable application and the definition of a point of input for the location-based application component. In this regard, the point of input can include at least one user interface control accepting data input of structured data. Finally, the deployable application is uploadable to a deployable application repository over a computer communications network for deployment to requesting mobile devices over the computer communications network.

It is apparent that numerous innovations for wireless devices for detecting, and/or identifying, and/or monitoring, and/or warning, and/or notifying remote environmental conditions have been provided in the prior art, which are adapted to be used. Furthermore, even though these innovations may be suitable for the specific individual purposes to which they address, nevertheless, they would not be suitable for the purposes of the embodiments of the present invention as heretofore described
because . . .
   unlike the current commercial chemical detection devices, the mobile hand-held chemical detection device of the embodiments of the present invention is miniature in size, provides an advance communication warning system accessed by smart devices, e.g., a smartphone, a tablet, and a laptop, and provides an affordable low-cost detection device for consumers. For instance, comparing the commercial hazardous vapor warning LCD 3.3¹ and Nose Gas Sensor² devices to the mobile CHCD device of the embodiments of the present invention, the LCD 3.3 and Nose Gas Sensor devices are designed as one unit with an LCD screen used for displaying gas concentration levels and are not capable of communicating with other smart devices. Additionally, both devices are not small and not cost effective for general consumers. Furthermore, many other commercial detection devices have similar features as the LCD 3.3 and Nose Gas Sensor. This is why the mobile CHCD device of the embodiments of the present invention is a detection device for an advanced warning system to the public. ¹ Smith Detection Group, [retrieved: March, 2012] http://www.smithsdetection.com/1023_{—}4601.php² University of Illinois, [retrieved: March, 2012] http://www.futurity.org/science-technology/sensor-sniffs-out-shoe-bombs/

Furthermore, in today's society, there is a challenge to detect and to avoid exposure to harmful and lethal chemicals. This remains an issue to public health, and has not been addressed adequately. To address this challenging issue, there is provided the mobile CHCD device of the embodiments of the present invention that can detect harmful and lethal chemical gases, such as NO₂, N₂, CO, CO₂, LPG, CH₄, CNG, C₂H₅OH, NH₃, H₂, and others, in public and in private gathering places.

The mobile CHCD device of the embodiments of the present invention can relay information of the chemical concentration levels detected to a smartphone or a tablet or a laptop in any place at any time. Applications of the mobile CHCD device of the embodiments of the present invention include detection of harmful gases in public and in private gathering places, such as subway stations, shopping malls, airports, and residential houses. Additionally, the mobile CHCD device of the embodiments of the present invention provides an alternative and affordable resource for people to have and use as an advance warning system within the proximity of dangerous areas.

### 3. SUMMARY OF THE INVENTION

Thus, an object of the embodiments of the present invention is to provide a mobile custom-made hand-held chemical detection device, which avoids the disadvantages of the prior art.

Briefly stated, another object of the embodiments of the present invention is to provide a mobile custom-made hand-held chemical detection device interfacing with a smart device. The device includes at least one sensor, a microcontroller, and a Bluetooth module. The at least one sensor detects an associated chemical and generates information in response thereto so as to form chemical detection information. The microcomputer is operatively connected to the at least one sensor and processes the chemical detection information therefrom so as to form processed chemical detection information. The Bluetooth module is operatively connected to the microcontroller and the smart device, and communicates the processed chemical detection information from the microcontroller to the smart device for interpretation.

The novel features considered characteristic of the embodiments of the present invention are set forth in the appended claims. The embodiments of the present invention themselves, however, both as to their construction and to their method of operation together with additional objects and advantages thereof will be best understood from the following description of the embodiments of the present invention when read and understood in connection with the accompanying figures of the drawing.

### 4. BRIEF DESCRIPTION OF THE FIGURES OF THE DRAWING

The figures of the drawing are briefly described as follows:

FIG. 1 is a schematic diagram of a chemical gas sensor of the mobile custom-made hand-held chemical detection device of the embodiments of the present invention;

FIG. 2 is a schematic diagram of the interfacing of a chemical gas sensor of the mobile custom-made hand-held chemical detection device of the embodiments of the present invention shown in FIG. 1;

FIG. 3 is a schematic diagram of the hardware of the mobile custom-made hand-held chemical detection device of the embodiments of the present invention;

FIG. 4 is a photograph of the mobile custom-made hand-held chemical detection device of the embodiments of the present invention;

FIG. 5 are photographs of the design stages of the mobile custom-made hand-held chemical detection device of the embodiments of the present invention;

FIG. 6 are assembly views of the mobile custom-made hand-held chemical detection device of the embodiments of the present invention;

FIG. 7 is a computer rendering of the mobile custom-made hand-held chemical detection device of the embodiments of the present invention;

FIG. 8 is a photograph of a physical prototype of the mobile custom-made hand-held chemical detection device of the embodiments of the present invention;

FIG. 9 is a block diagram of the Bluetooth wireless communication of the mobile custom-made hand-held chemical detection device of the embodiments of the present invention;

FIG. 10 is a flowchart of the wireless interface and communications of the mobile custom-made hand-held chemical detection device of the embodiments of the present invention;

FIG. 11 is a block diagram of the calibration procedure;

FIG. 12 is a chart of gas concentration in PPM and symptoms;

FIG. 13 is a graph of gas concentration in PPM;

FIG. 14 is a photograph of indoor testing using a laptop;

FIG. 15 is a photograph of indoor testing using a smartphone;

FIG. 16 is a chart of data from preliminary indoor tests;

FIG. 17 is graphs of sensor resistance for indoor tests;

FIG. 18 is a photograph of outdoor testing using a tablet;

FIG. 19 is a photograph of outdoor testing using a smartphone;

FIG. 20 is a chart of data from the preliminary outdoor tests; and

FIG. 21 are graphs of sensor resistance for outdoor tests.

### 5. DETAILED DESCRIPTION OF THE PREFERRED EMBODIMENTS

A. Introductory.

The mobile custom-made hand-held chemical detection device of the embodiments of the present invention can detect harmful and lethal chemical gases in public and in private gathering places, and is capable of communicating to portable devices, such as smartphones or tablets or laptops through the use of Bluetooth technology.

B. Chemical Gas Sensors.

Currently, there are many different types of harmful chemicals and gases, such as NO₂, N₂, CO, CO₂, LPG, CH₄, CNG, C₂H₅OH, H₂ and others that can harm innocent people and could give serious negative environmental impact on the planet we live on. To prevent the loss of innocent human lives, effective detection and hand-held monitoring systems need to be developed. The mobile CHCD device of the embodiments of the present invention detects many kinds of harmful chemical gases in the air and on the ground through the use of various chemical gas sensors, provides an advanced warning system, and alerts the general public through smart devices. This would reduce human casualties, environmental destruction, and property loss.

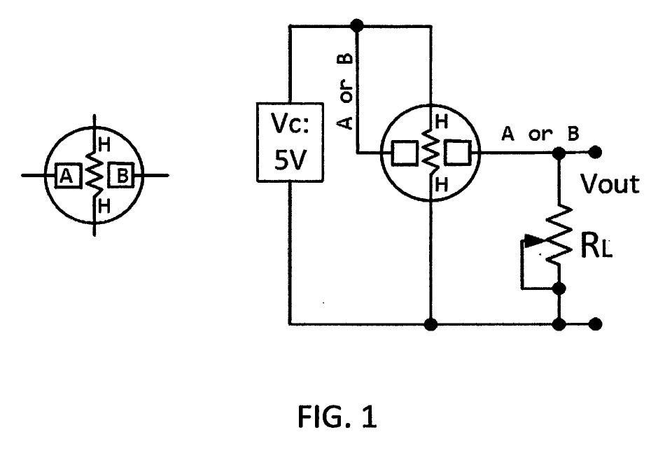

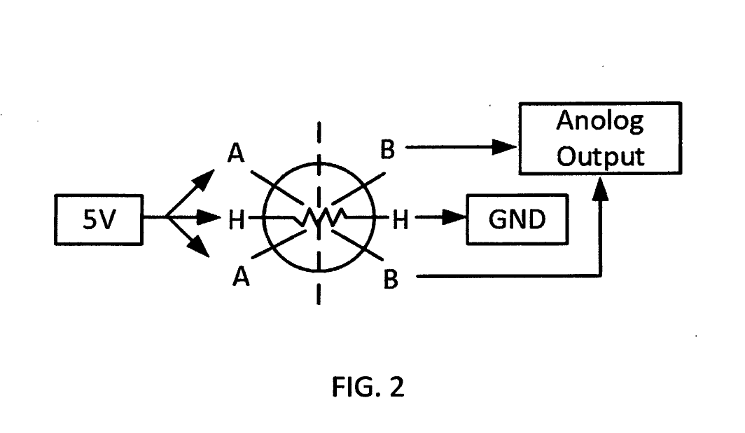

Many gas sensors use a heater to detect certain gases. In general, many of these gas sensors have a similar schematic diagram,³ as shown in FIG. 1, which is a schematic diagram of a chemical gas sensor of the mobile custom-made hand-held chemical detection device of the embodiments of the present invention, there are 6 pins coming out of the gas sensor itself. Some chemical gas sensors, however, have only 3 or 4 pins. 5 volts can be supplied to pins A H A, whereas, both pins B can be used as an analog output signal. The other pin H, between both pins B, can be used as a ground (GND) pin. This is illustrated in FIG. 2, which is a schematic diagram of the interfacing of a chemical gas sensor of the mobile custom-made hand-held chemical detection device of the embodiments of the present invention shown in FIG. 1. Then the data from an analog output signal is incorporated into specially designed software codes capable of detecting a wide range of harmful and lethal chemical gases by using different types of gas sensors. ³ Parallax Inc. [retrieved: February, 2012] http://www.parallax.com/Portals/O/Downloads/docs/prod/sens/MQ-7.pdf

In terms of the working principle of a gas sensor for CO₂, as an example, it takes on the solid electrolyte cell principle and is composed by the following solid cells:

AirAu|NASICON∥carbonate|Au, air, CO₂⁴

⁴ Parallax Inc., [retrieved: February, 2012] http://www.paralax.com/Store/Sensors/GasSensors/tabid/843/CategoryID/List/0/SortField0/Level/a/ProductID/598/Default.aspx

When a CO₂ sensor is exposed to a CO₂ environment, it will have an electrochemical reaction with the following reaction equations⁵: ⁵ 8085 Projects, Info, [retrieved: February, 2012] http://www.8086projects.info/default.aspx

Cathodic reaction equation:

2Li++CO₂+1/2O₂+2e−=Li₂CO₃

Anodic reaction equation:

2Na+1/2O₂+2e−=Na₂O

Overall chemical reaction equation:

Li₂CO₃+2Na+=Na₂O+2Li++CO₂

As a result of the electrochemical reaction, according to Neste equation (Nernst), the process produces the following electromotive force (EMF):

EMF=Ec−®×T)/(2F)ln(P(CO₂))

where:
PCO₂ is the partial pressure of CO₂;
Ec is a constant:
R is the gas constant;
T is temperature in Kelvin; and
F is the Faraday constant.

As shown in FIG. 1, the sensor heating voltage is supplied from another circuit. When its surface temperature is high enough, the sensor is equal to a cell, its two sides output a voltage signal, and its result will be according to Nernst's equation. In sensor testing, the impedance of the amplifier should be within 100-1000 GΩ. Its testing current should be controlled below 1 pA.

C. Configuration of the Mobile Custom-Made Hand-Held Chemical Detection Device of the Embodiments of the Present Invention.

The set up pins of the gas sensor shown in FIG. 1 provide some idea of how to hook up the hardware of the mobile custom-made hand-held chemical detection device of the embodiments of the present invention.

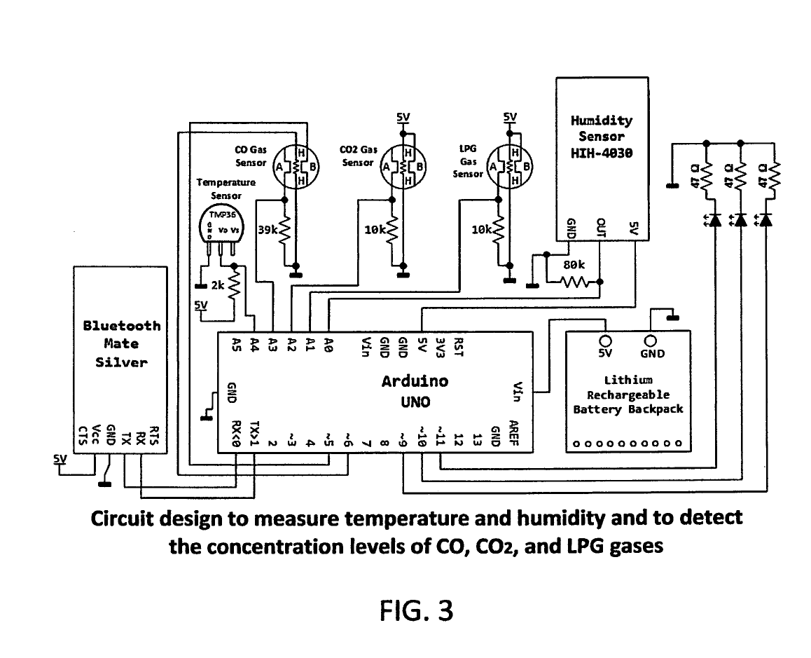

Using a Fritzing⁶ software program, the configuration of the mobile custom-made hand-held chemical detection device of the embodiments of the present invention can best be seen in FIG. 3, which is a schematic diagram of the hardware of the mobile custom-made hand-held chemical detection device of the embodiments of the present invention. ⁶ Fritzing Inc., [retrieved: January, 2012] http//www.10.fritzing.com/

From FIG. 3, the yellow, red, and black wires are used as data signal communication, voltage, and ground, respectively. The signal lines for the three sensors, i.e., CO gas, CO₂ gas, and LPG gas, are connected to an Arduino microcontroller analog pins A1, A3, and A4. In addition to the three gas sensors shown in FIG. 3, temperature and humidity sensors are added to the mobile custom-made hand-held chemical detection device of the embodiments of the present invention for monitoring the effect of data acquisition in correlation to the three gas sensors. Whereas, the red and black lines across the breadboard shown in FIG. 3 are connected to Anduino 5V and ground pins, respectively. The yellow and white wires from the Bluetooth module are used as the transceivers and are connected to Arduino pins TX (transmitter—yellow wire) and RX (receiver—white wire). The use of a Bluetooth module provides the wireless communication lines between the mobile custom-made hand-held chemical detection device of the embodiments of the present invention and a smartphone or a tablet or a laptop. Also, for instance, the three color LEDs indicate the CO gas concentration levels. Green, orange, and red color LEDs indicate the least, medium, and highest PPM (parts per million), respectively. PPM is used to measure the concentration of chemical gas. Hence, this makes the mobile custom-made hand-held chemical detection device of the embodiments of the present invention an embedded mobile and hand-held device in monitoring the surrounding areas of one's presence.

D. Example I—Prototyping the Mobile Custom-Made Hand-Held Chemical Detection Device of the Embodiments of the Present Invention.

Based on the hardware schematic of the mobile custom-made hand-held chemical detection device of the embodiments of the present invention shown in FIG. 2, a prototype was created as shown in FIG. 3.

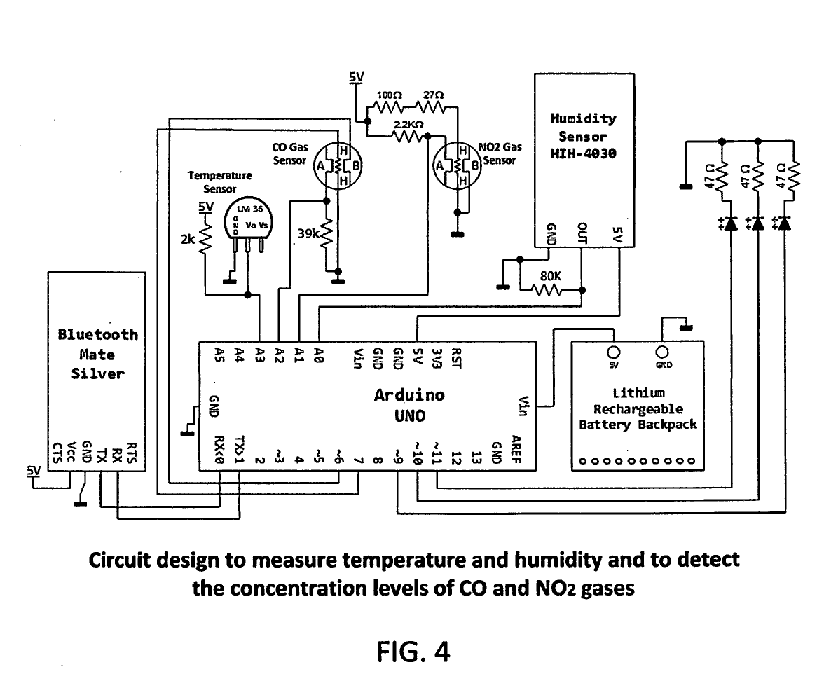

As seen in FIG. 4, which is a photograph of the mobile custom-made hand-held chemical detection device of the embodiments of the present invention, the mobile custom-made hand-held chemical detection device of the embodiments of the present invention is being used for testing CO (Carbon Monoxide) concentration levels along with temperature and humidity.

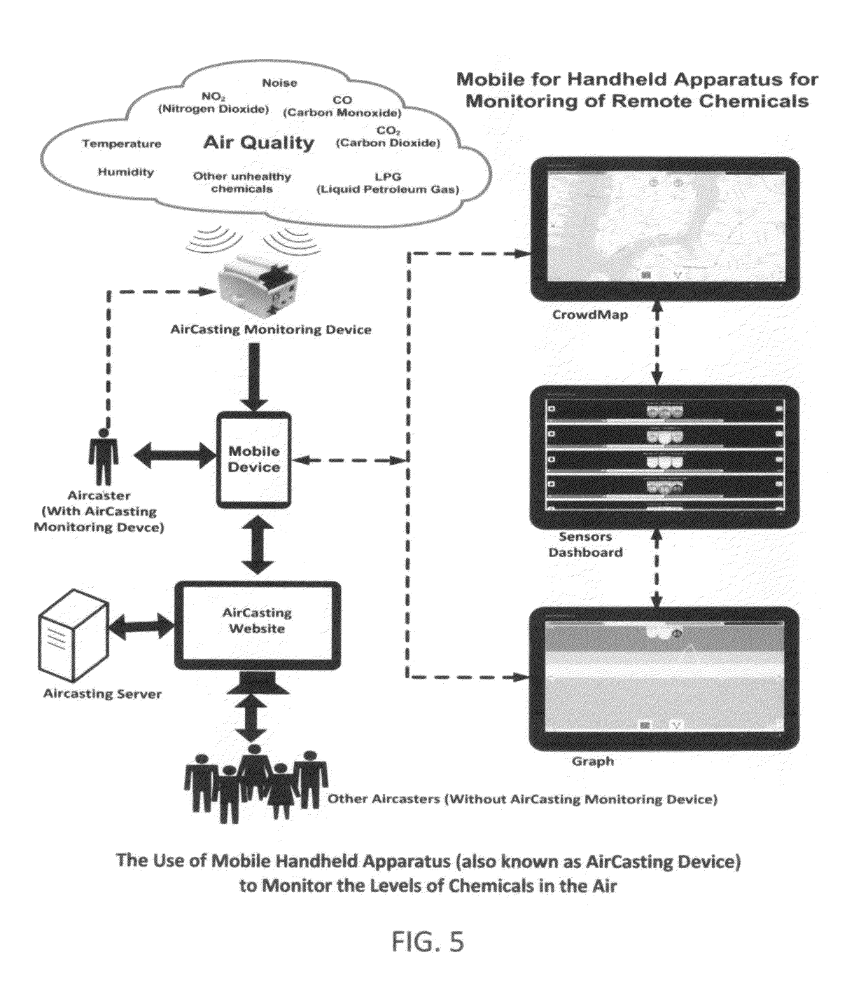

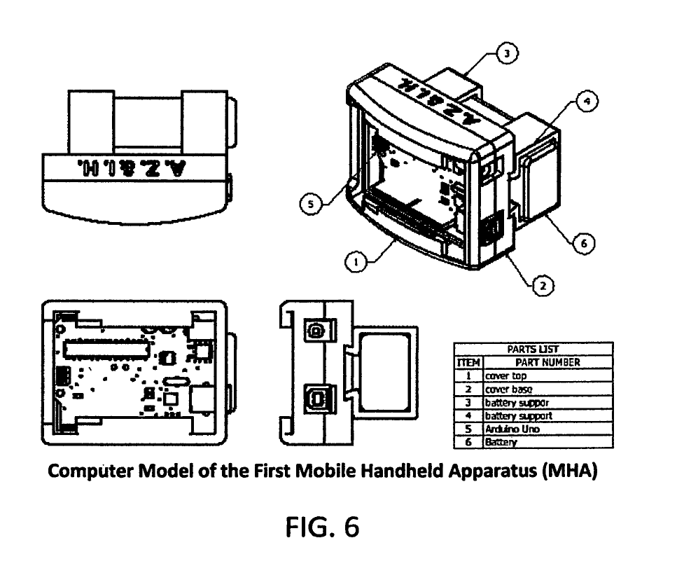

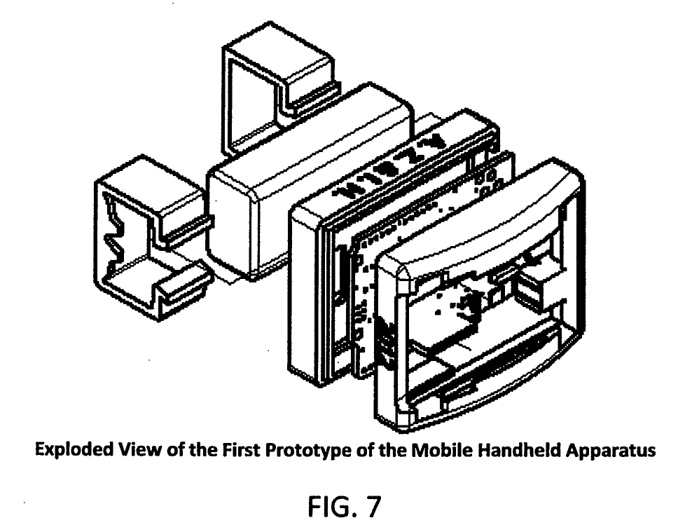

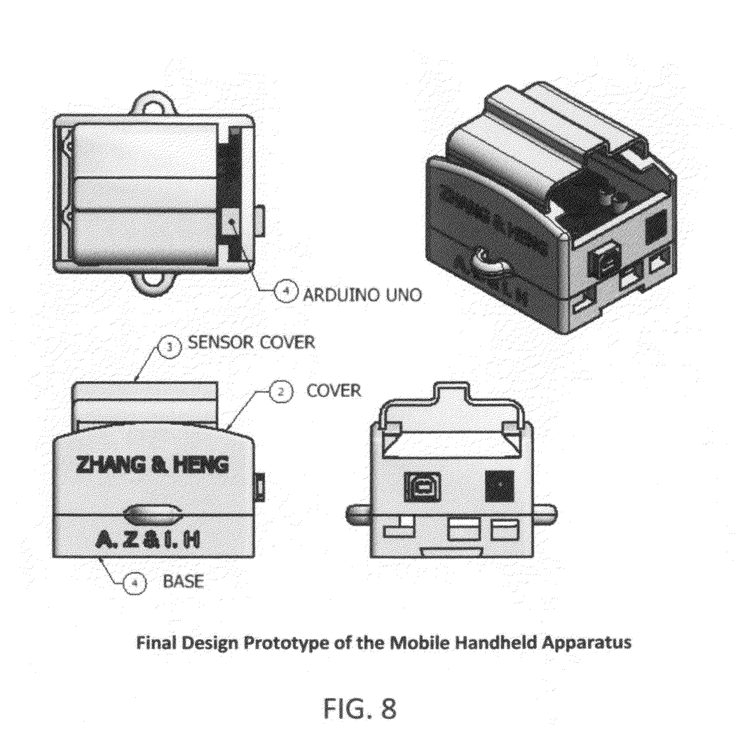

As shown in FIG. 5, which are photographs of the design stages of the mobile custom-made hand-held chemical detection device of the embodiments of the present invention, the case of the mobile custom-made hand-held chemical detection device of the embodiments of the present invention holds an Arduino UNO microcontroller and electronic components, i.e., sensors, resistors, LEDs, and Bluetooth module, with breadboard. To accomplish this a computer module of the mobile custom-made hand-held chemical detection device of the embodiments of the present invention was created using Autodesk Inventor⁷ software. FIG. 6, which are assembly views of the mobile custom-made hand-held chemical detection device of the embodiments of the present invention, illustrates a model of the mobile custom-made hand-held chemical detection device of the embodiments of the present invention, while FIG. 7 is a computer rendering of the mobile custom-made hand-held chemical detection device of the embodiments of the present invention. Then a physical prototype was made using a 3D rapid prototyping machine as shown in FIG. 8, which is a photograph of a physical prototype of the mobile custom-made hand-held chemical detection device of the embodiments of the present invention. The electronic components are soldered on the back side of the breadboard. Upon completing soldering all of the electronic components on the breadboard, the completed breadboard was slid into the orange case. This completes the design stages of the physical prototype of the mobile custom-made hand-held chemical detection device of the embodiments of the present invention. ⁷ Autodesk, Inc. [retrieved: January, 2012] http://www.autodesk.com

E. Wireless Communication.

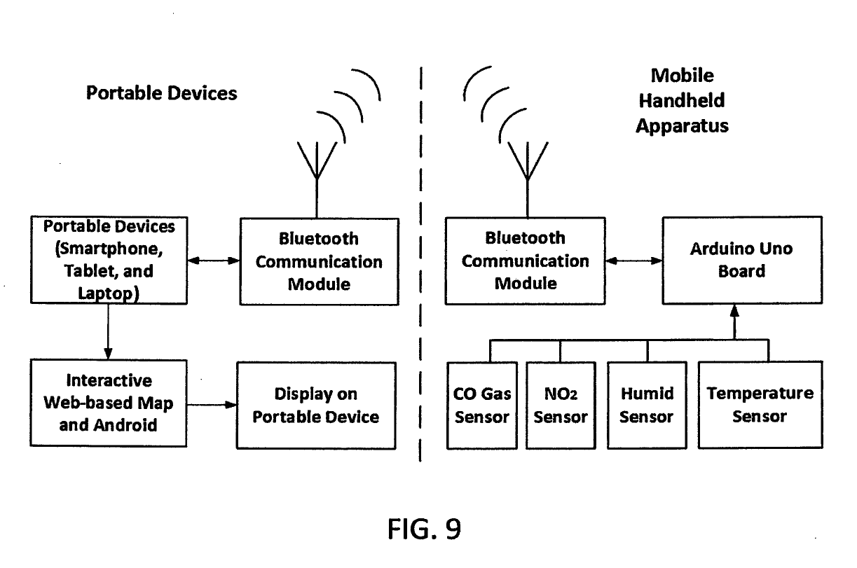

As shown in FIG. 9, which is a block diagram of the Bluetooth wireless communication of the mobile custom-made hand-held chemical detection device of the embodiments of the present invention, the mobile custom-made hand-held chemical detection device of the embodiments of the present invention is capable of communicating to portable devices, such as a smartphone or a tablet or a laptop through the use of Bluetooth technology. Bluetooth wireless technology is based on the IEEE 802.15 standard. Bluetooth was developed to replace the cables that were connected to desktop and portable computers, mobile phones, hand-held devices, computer accessories, and peripheral electronic devices.⁸ Thus, the use of Bluetooth wireless communication enables the users to retrieve the data from the chemical gas sensors and at the same time display gas concentration levels on the portable devices. ⁸ I. Heng, F. Zia, and A. Zhang, “Wired and wireless Port Communication.” In proceedings of the 118^{th} Annual ASEE Conference and Exposition, Jun. 26-29, 2011. Vancouver, British Columbia, Canada.

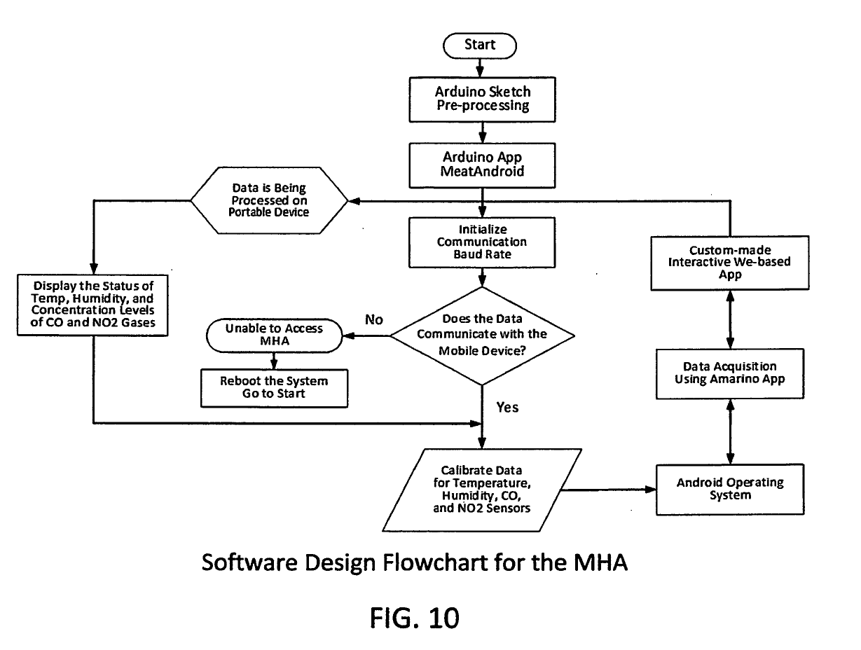

To make the mobile custom-made hand-held chemical detection device of the embodiments of the present invention communicate and interface wirelessly with portable devices, i.e., a smartphone, a tablet, and a laptop, as illustrated in FIG. 9, the programming source codes must be introduced to provide access of communication and interface between devices. First, the source codes are written in Arduino sketch⁹ to communicate and interface with the electronic components, such as CO gas sensor, temperature sensor, humidity sensor, and LEDs, as shown in FIG. 5. Upon the success of interfacing with the sensors and LEDs in Arduino sketch, the Android library MeetAndroid is imported to the Arduino library folder, so that the data acquired from the Arduino Serial Monitor is sent to the Amarino Application (App) program. The Android library MeetAndroid is part of Amarion driver device that is required to be imported into the library folder of Arduino sketch. The Amarino program¹⁰ is a freeware program that incorporates a plug-in mechanism that allows programmers and developers to integrate their events into Amarino. Then, this provides a gateway to communicate with smartphones and tablets based on the Android open source operating system. FIG. 10, which is a flowchart of the wireless interface and communications of the mobile custom-made hand-held chemical detection device of the embodiments of the present invention, illustrates the details of communication between Arduino Sketch, Amarino App, and Android operating system. ⁹ Arduino, [retrieved: January, 2012] http://arduino.cc/en/¹⁰ Bonifax Kaufmann, [retrieved: March, 2012] http://www.amarino-toolkit.netindex.php/home.html

F. Example II—CO Gas Sensor Calibration.

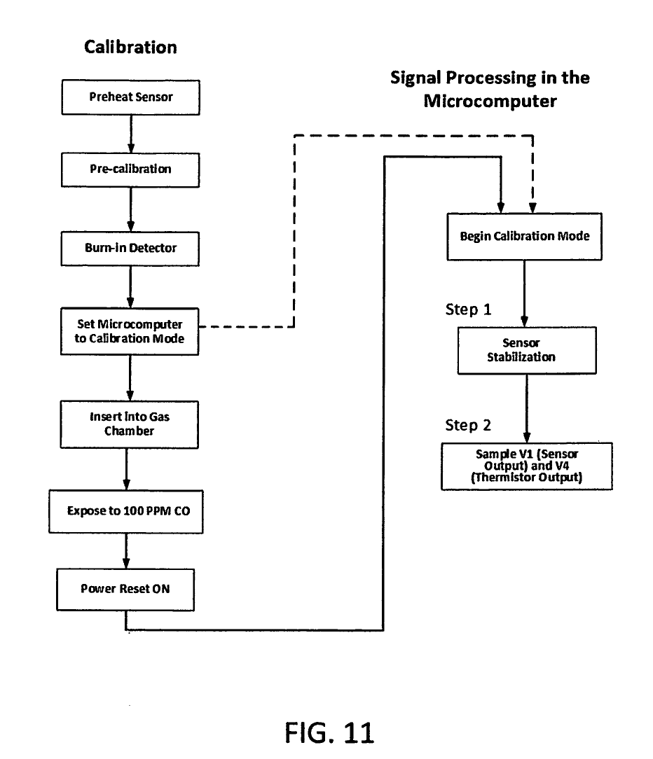

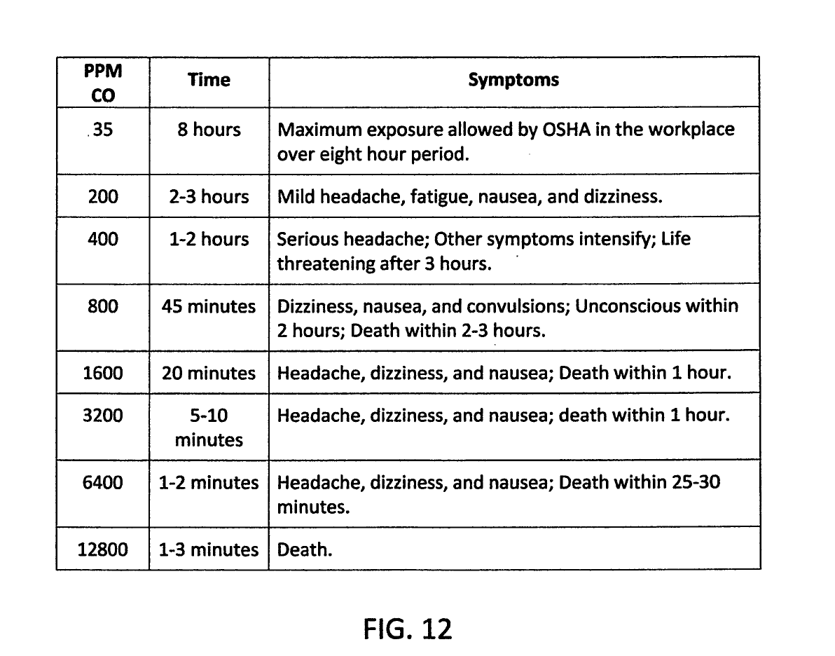

Using the mobile custom-made hand-held chemical detection device of the embodiments of the present invention shown in FIG. 4, the raw analog signal data was acquired from the CO gas monitor. Then the raw data must be calibrated with respect to the analog data. For instance, taking all factors, such as the type of sensor and the conditions of the application into consideration, the proposed calibration procedure is based on the block diagram of FIG. 11, which is a block diagram of the calibration procedure. Based on the flowchart of FIG. 11, 100 PPM is exposed and used for the calibration standard. Applications of how safe and unsafe PPM for CO gas can be seen in FIG. 12, which is a chart of gas concentration in PPM and symptoms. For instance, 200 PPM would have symptoms of mild headache, fatigue, nausea, and dizziness in two to three hours.¹¹ ¹¹ About.com Biology, [retrieved: March, 2012] http://www.biology.about.com/od/molecularbiology/a/carbon_monoxide.htm

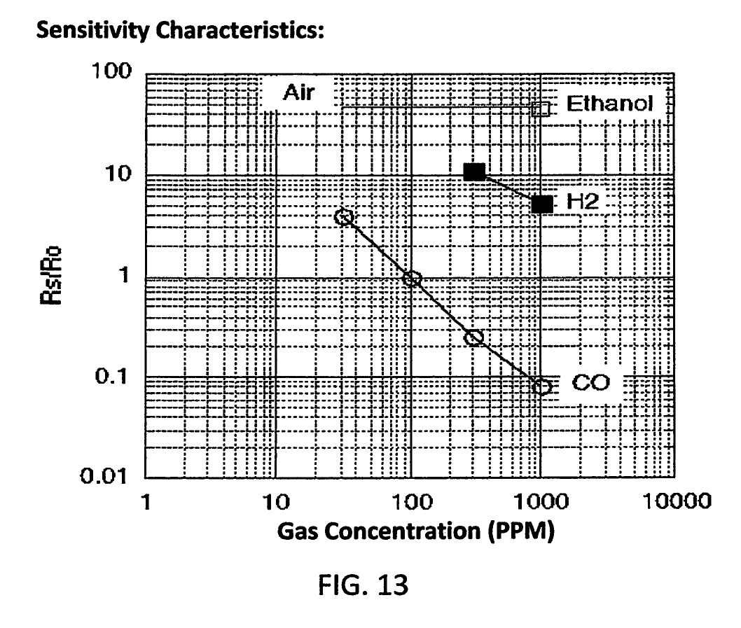

In addition to the block diagram of FIG. 11, the gas concentration chart¹² in FIG. 13, which is a graph of gas concentration in PPM, is used as part of the calibration for the CO concentration in PPM. FIG. 13 represents typical sensitivity characteristics of CO concentration levels. The Y-axis is indicated as the sensor resistance ratio (Rs/Ro),¹³ which is defined as follows: ¹² Figaro USA Inc., [retrieved: April, 2012]¹³ See footnote 12.

Rₛ=Sensor resistance of displayed gases at various concentrations; and

Rₒ—Sensor resistance in 100 PPM CO

Another way of looking at Rₒ is the level of exposed gas to the sensor in clean air. For instance, if we pour 100 PPM gas into a container with a confined space, what would a Rₛ sensor read? It may read 98 PPM or 102 PPM.

The formula¹⁴ for defining the sensor resistance Rₛ is as follows as equation (1): ¹⁴ A. Sri-on, S. Sanongraj, and M. Pusayatanont, “A Simple Microcontroller Circuit for Carbon Monoxide Sernsor.” The 8^{th} Asian-Pacific Regional Conference on Practical Environmental Technologies, Ubon Ratchathani University, Ubonratchathani, Thailand, Mar. 24-27, 2010

Rₛ=((V_{c}×R_{L})/Vₒᵤₜ)−R_{L}

From equation (1), V_{c} is the voltage input, and it is 5 Volts from the Arduino microcontroller embedded in the mobile custom-made hand-held chemical detection device of the embodiments of the present invention. R_{L} is the load resistance (in this case, we use 39 kΩ) that is connected to the CO gas sensor. Vₒᵤₜ is a voltage signal from the CO gas sensor, which varies depending on the amount of CO concentration in PPM. Then the value of Rₛ in equation (1) changes according to the amount of CO gas present, and as seen in FIG. 13, the typical range for CO gas concentration is from 30 to 1000 PPM. If Rₛ resistance value is the same as Rₒ resistance value, it means that 100/100=1, which correlates to 100 PPM in FIG. 13. In theory, Rₒ represents the X axis in FIG. 13 if conditions are perfect.

G. Example III—Preliminary Testing and Results.

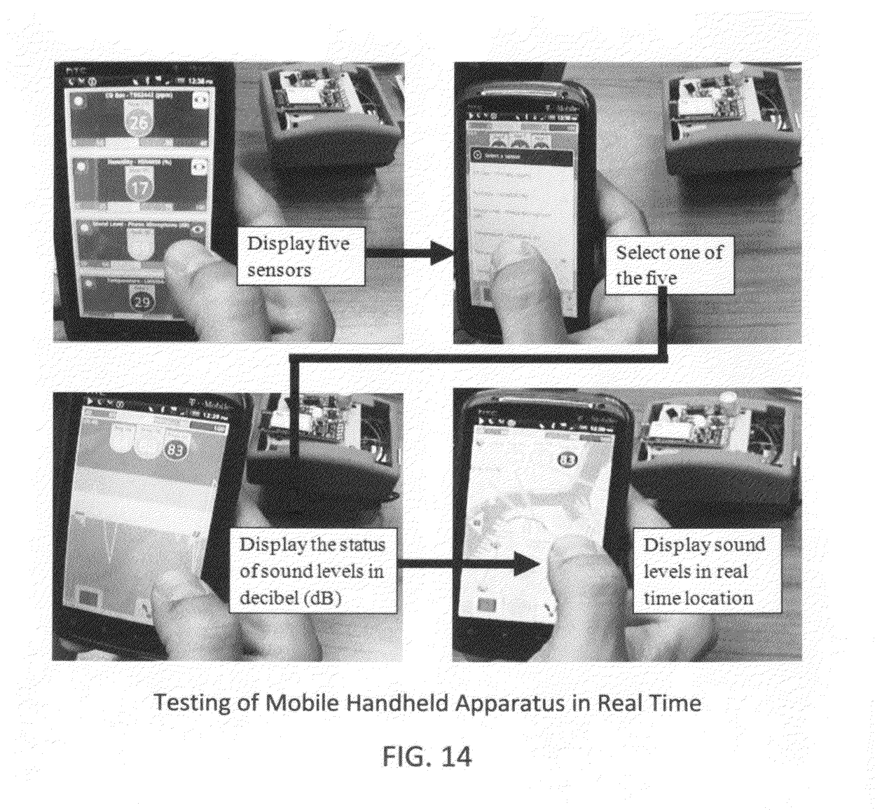

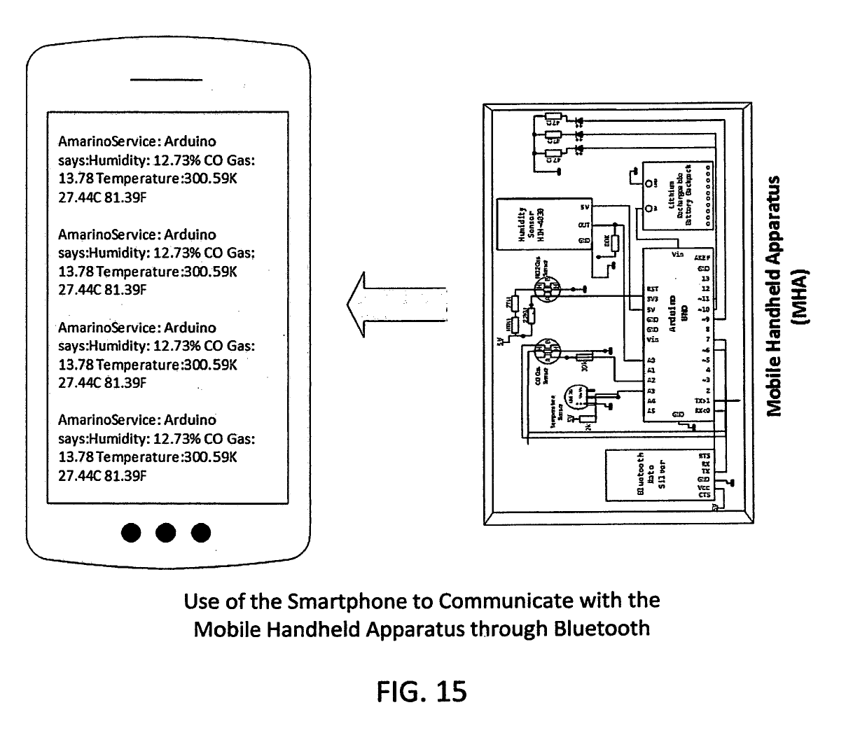

We have done several tests of the mobile custom-made hand-held chemical detection device of the embodiments of the present invention with a smartphone, a laptop, and a tablet. The indoor tests are shown in FIG. 14, which is a photograph of indoor testing using a laptop, and FIG. 15, which is a photograph of indoor testing using a smartphone.

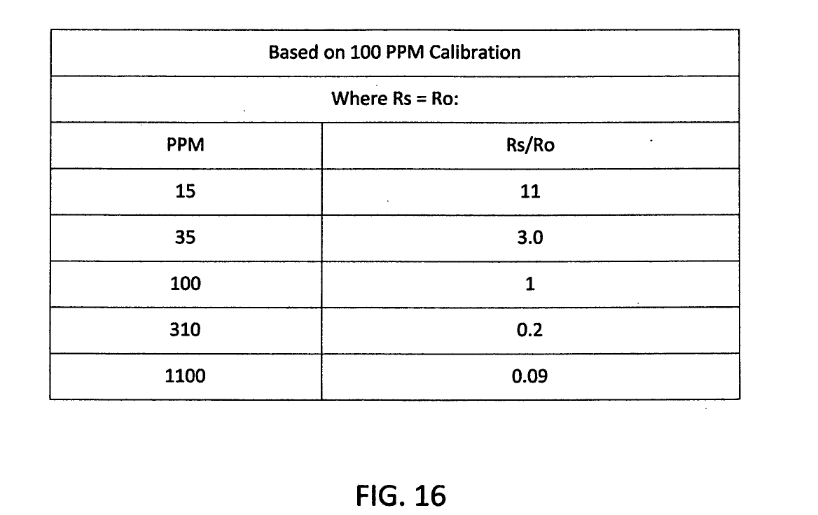

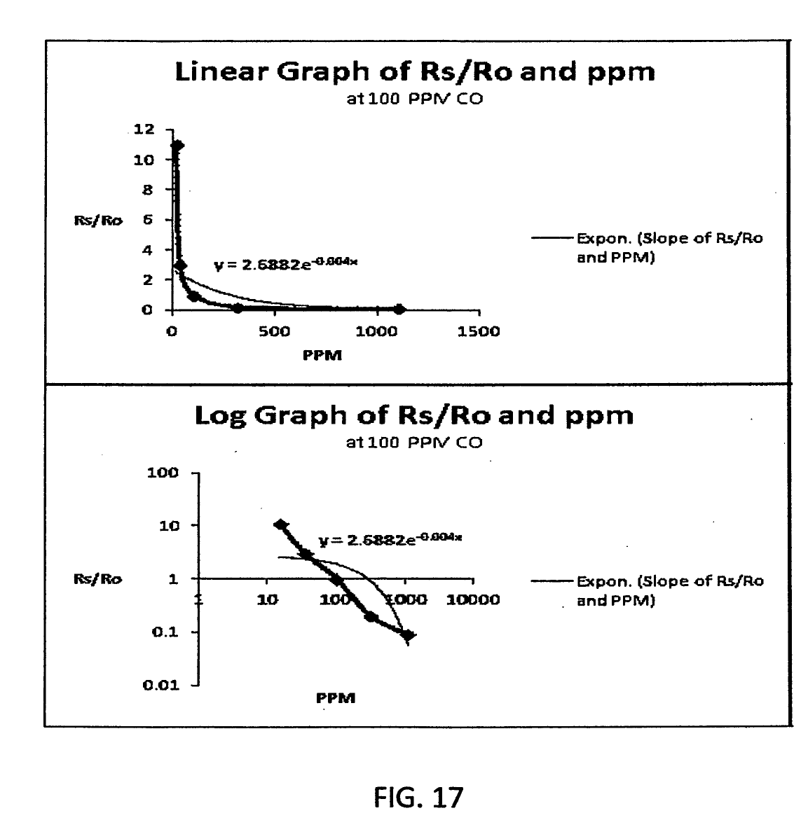

The preliminary data from the indoor tests can be seen in FIG. 16, which is a chart of data from preliminary indoor tests. The data is then tabulated in Excel spreadsheet. Then the data in FIG. 16 is plotted in Excel chart. The chart can be seen in FIG. 17, which is graphs of sensor resistance for indoor tests. And it is used to tell the sensitivity characteristics of CO concentration levels.

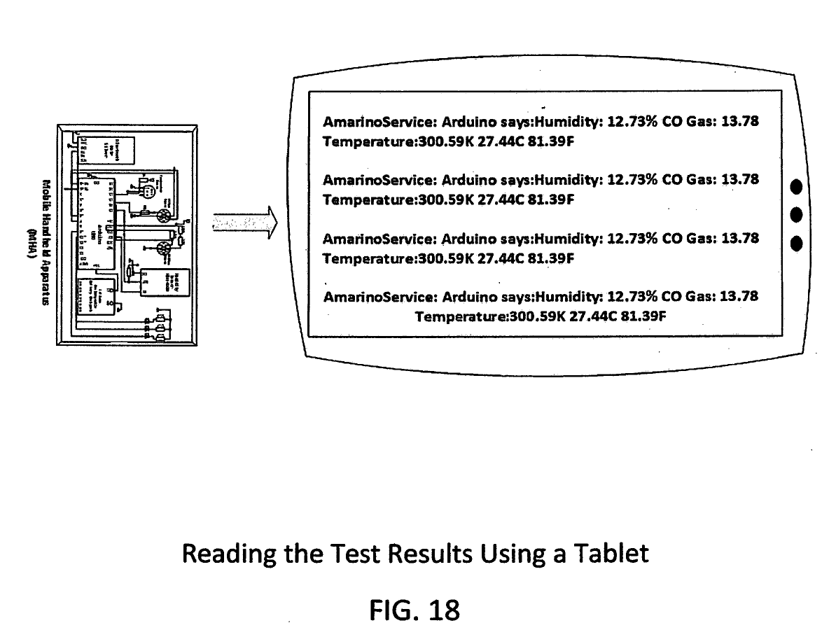

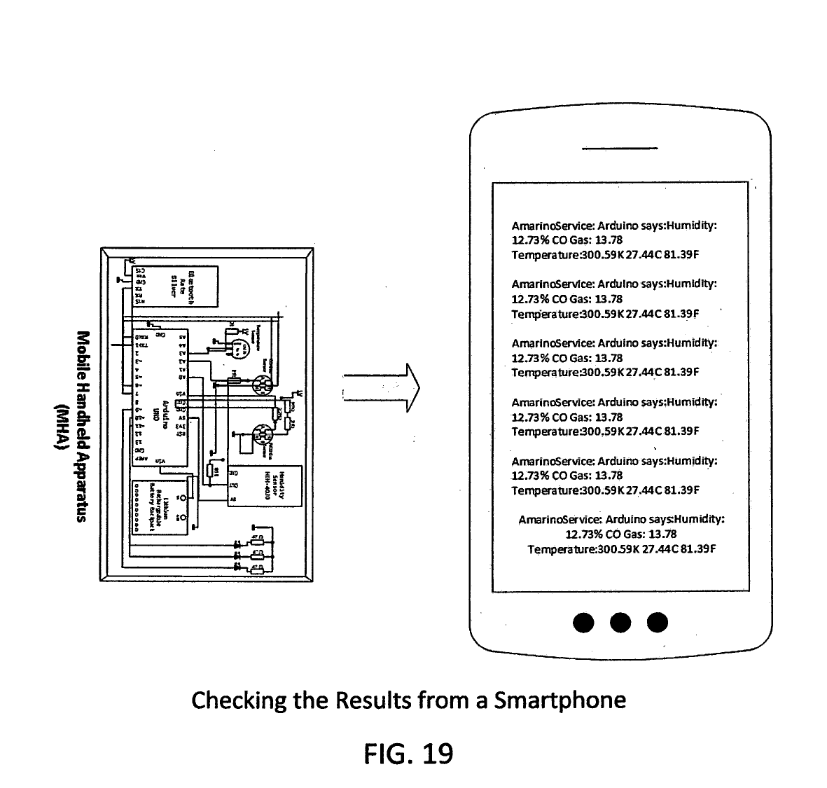

Similarly, the outdoor tests of the mobile custom-made hand-held chemical detection device of the embodiments of the present invention (without the case) were performed with a tablet and a smartphone. The test was done by placing the mobile custom-made hand-held chemical detection device of the embodiments of the present invention behind a car's exhaust pipe while the car engine was on, as shown in FIG. 18, which is a photograph of outdoor testing using a tablet, and FIG. 19, which is a photograph of outdoor testing using a smartphone.

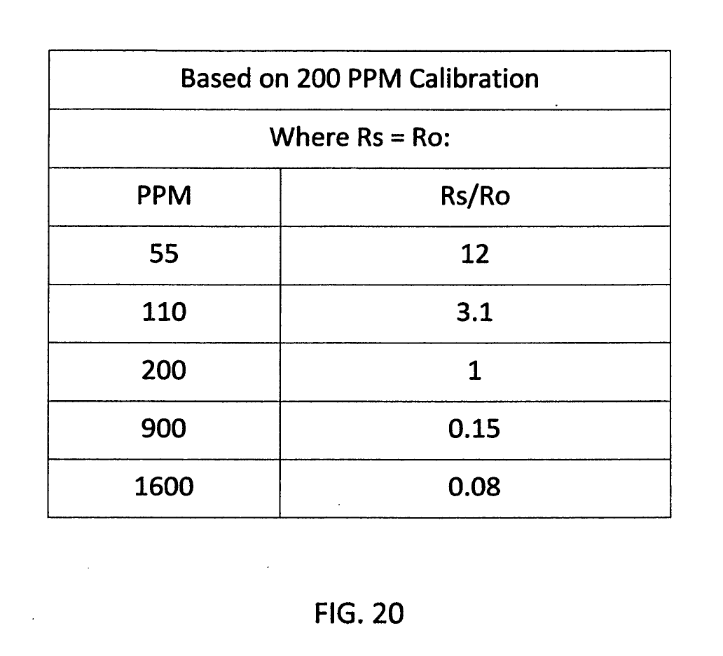

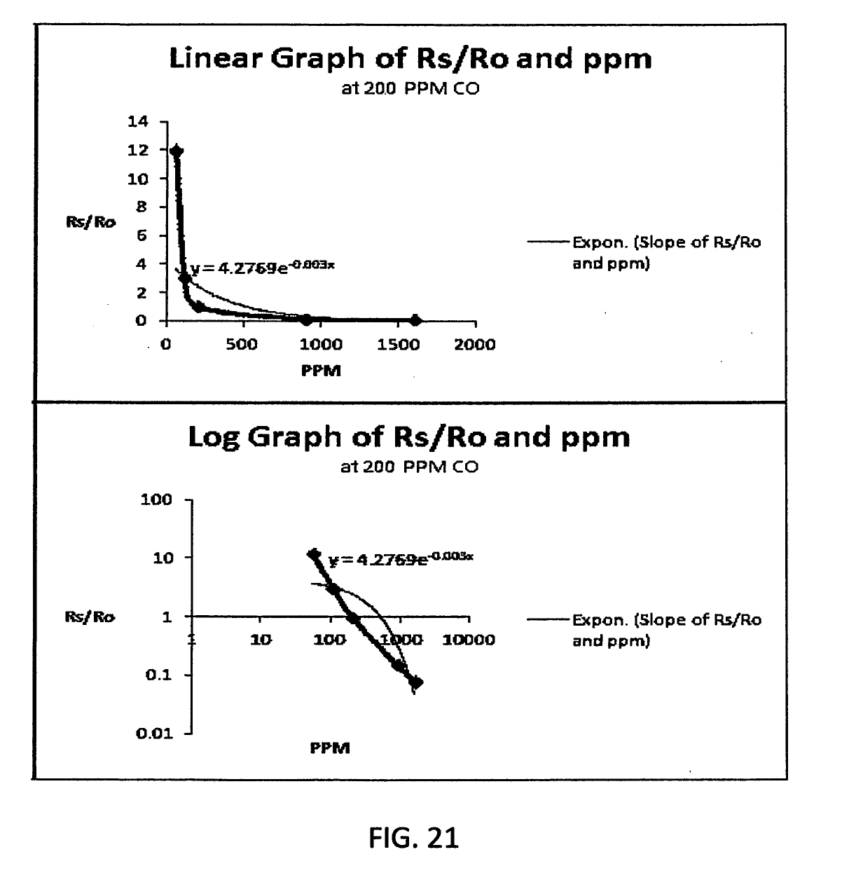

The preliminary data from the outdoor testing can be seen in FIG. 20, which is a chart of data from the preliminary outdoor tests. Then the data in FIG. 20 is plotted in Excel chart and can be seen in FIG. 21, which are graphs of sensor resistance for outdoor tests.

Two scenarios were conducted on the mobile custom-made hand-held chemical detection device of the embodiments of the present invention using a smartphone, a tablet, and a laptop. Of the two scenarios, the worst case was found when the mobile custom-made hand-held chemical detection device of the embodiments of the present invention was placed behind a car's exhaust pipe while the car engine was running. These results are encouraging and show that the mobile custom-made hand-held chemical detection device of the embodiments of the present invention provides reliable data to determine the chemical concentration PPM levels. Note that when performing outdoor tests of the mobile custom-made hand-held chemical detection device of the embodiments of the present invention, wind, temperature, and humidity were taken into consideration.

H. Summary and Conclusion.

The mobile custom-made hand-held chemical detection device of the embodiments of the present invention assists and provides significant impact to society in terms of reducing the potential loss of human life through detection and prevention. The mobile custom-made hand-held chemical detection device of the embodiments of the present invention provides an early warning system if there is possible exposure of harmful and lethal chemical concentration levels within distance. This early detection and prevention would save many lives from harmful chemical gases. Thus, the mobile custom-made hand-held chemical detection device of the embodiments of the present invention becomes an analytics engine capable of picking up emergent patterns in human environments and biology.

The mobile custom-made hand-held chemical detection device of the embodiments of the present invention is a low-cost miniature detection device that would provide crucial instant information of chemical detection and prevent the loss of human life. This crucial information of sensing and detecting the quality of the air becomes possible with the aid of modern technologies, e.g., a smartphone, a tablet, and a laptop. Hence, the mobile custom-made hand-held chemical detection device of the embodiments of the present invention, along with modern technologies, provide an alternative affordable resource for people to have and use to identify invisible harmful chemicals at early warning stages and would possibly lead to save many lives.

I. Impressions.

It will be understood that each of the elements described above, or two or more together, may also find a useful application in other types of constructions differing from the types described above.

While the embodiments of the present invention have been illustrated and described as embodied in a mobile custom-made hand-held chemical detection device, nevertheless, they are not limited to the details shown, since it will be understood that various omissions, modifications, substitutions, and changes in the forms and details of the embodiments of the present invention illustrated and their operation can be made by those skilled in the art without departing in any way from the spirit of the embodiments of the present invention.

Without further analysis, the foregoing will so fully reveal the gist of the embodiments of the present invention that others can by applying current knowledge readily adapt them for various applications without omitting features that from the standpoint of prior art fairly constitute characteristics of the generic or specific aspects of the embodiments of the present invention.

The invention claimed is:

1. A mobile custom-made hand-held chemical detection device for interfacing with a smart device, comprising:
a) at least one sensor;
b) a microcontroller; and
c) a Bluetooth module;
wherein said at least one sensor is for chemical detection;
wherein said at least one sensor generates information in response to the chemical detection so as to form chemical detection information;
wherein said microcomputer is operatively connected to said at least one sensor;
wherein said microcontroller processes said chemical detection information generated by said at least one sensor so as to form processed chemical detection information;
wherein said Bluetooth module is operatively connected to said microcontroller;
wherein said Bluetooth module is for being operatively connected to the smart device; and
wherein said Bluetooth module communicates said processed chemical detection information from said microcontroller to the smart device for interpretation.

2. The device of claim 1, wherein said at least one sensor includes an NO₂ sensor.

3. The device of claim 1, wherein said at least one sensor includes an N₂ sensor.

4. The device of claim 1, wherein said at least one sensor includes a CO sensor.

5. The device of claim 1, wherein said at least one sensor includes a CO₂ sensor.

6. The device of claim 1, wherein said at least one sensor includes an LPG sensor.

7. The device of claim 1, wherein said at least one sensor includes a CH₄ sensor.

8. The device of claim 1, wherein said at least one sensor includes a CNG sensor.

9. The device of claim 1, wherein said at least one sensor includes a C₂H₅OH sensor.

10. The device of claim 1, wherein said at least one sensor includes an H₂ sensor.

11. The device of claim 1, wherein said at least one sensor includes a temperature sensor.

12. The device of claim 1, wherein said at least one sensor includes a humidity sensor.

13. The device of claim 1, wherein said microcontroller is an Arduino microcontroller.

14. The device of claim 1, wherein the smart device includes a smartphone.

15. The device of claim 1, wherein the smart device includes a tablet.

16. The device of claim 1, wherein the smart device includes a laptop.

17. The device of claim 1, further comprising a breadboard.

18. The device of claim 1, further comprising an LED display.

19. The device of claim 18, wherein said LED display includes three differently colored LEDs for each at least one sensor.

20. The device of claim 19, wherein said three differently colored LEDs for each at least one sensor of said LED display indicates concentration levels.

21. The device of claim 20, wherein said concentration levels of said three differently colored LEDs for each at least one sensor of said LED display indicate least, medium, and highest PPM, respectively.

22. The device of claim 1, further comprising a case.

23. The device of claim 1, further comprising resistors.
